(19) United States
(12) Patent Application Publication
(10) Pub. No.: US 2006/0229504 A1
(43) Pub. Date: Oct. 12, 2006

(54) Methods and sytems for lifestyle management

(76) Inventor: Marvin R. Johnson JR., Atlanta, GA (US)

Correspondence Address: NEEDLE & ROSENBERG, P.C., SUITE 1000, 999 PEACHTREE STREET, ATLANTA, GA, 30309-3915, US

(21) Appl. No.: 11/399,727

(22) Filed: Apr. 7, 2006

Related U.S. Application Data
(60) Provisional application No. 60/669,493, filed on Apr. 8, 2005.

(51) Int. Cl. A61B 5/00

(52) U.S. Cl. 600/300; 128/921

(57) ABSTRACT

Disclosed are systems and methods for lifestyle management. These systems and method can comprise selecting a lifestyle, scanning a product consumed wherein a unique identifier associated with the product is stored, transferring the unique identifier to a database, receiving nutritional information associated with the unique identifier, and receiving dietary progress analysis based on the selected lifestyle and the product consumed. The lifestyle can be weight loss, weight gain, or weight maintenance.

### BACKGROUND OF THE INVENTION

“Dieting,” like American girth, is both big and rapidly growing. At any time, millions of people are both dieting and spending billions on a myriad of diet products and services, mostly with minimal success. While specific diets are debated, it is widely agreed that “sticking with it” is the number one issue in diet success. The pundits agree that tracking nutritional intake is a key factor in helping health or weight conscious persons “stick with it.”

### SUMMARY OF THE INVENTION

Disclosed herein is a system and method for lifestyle management that comprises receiving a selected lifestyle and receiving a unique identifier from a product that is scanned by a scanner. In one aspect, the unique identifier is associated with a product to be consumed by the user. In further aspects, nutritional information is associated with the unique identifier and dietary progress analysis is provided to the user based on the user selected lifestyle and the product(s) to be consumed. In alternative aspects, the lifestyle selected can be, for example and not meant to be limiting, weight loss, weight gain, or weight maintenance.

Related methods of operation are also provided. Other systems, methods, features, and advantages of the invention will be or become apparent to one with skill in the art upon examination of the following figures and detailed description. It is intended that all such additional systems, methods, features, and advantages be included within this description, be within the scope of the invention, and be protected by the accompanying claims.

### BRIEF DESCRIPTION OF THE DRAWINGS

The accompanying drawings, which are incorporated in and constitute a part of this specification, illustrate several aspects described below and together with the description, serve to explain the principles of the invention. Like numbers represent the same elements throughout the figures.

FIG. 1 is a block diagram illustrating an exemplary operating environment for performing the disclosed method.

FIG. 2 is a flowchart illustrating a method for lifestyle management.

FIG. 3 is a flowchart illustrating a method for lifestyle management.

FIG. 4 is a flowchart illustrating an exemplary method of the present invention.

FIG. 5 is an exemplary user interface for a weight goal calculator.

FIG. 6 is a flowchart representing exemplary consumption management.

FIG. 7 illustrates data relationships of the present invention.

### DETAILED DESCRIPTION OF THE INVENTION

The present invention can be understood more readily by reference to the following detailed description, examples, drawing, and claims, and their previous and following description. However, before the present devices, systems, and/or methods are disclosed and described, it is to be understood that this invention is not limited to the specific devices, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known embodiment. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used in the specification and the appended claims, the singular forms “a,” “an” and “the” comprise plural referents unless the context clearly dictates otherwise.

Ranges can be expressed herein as from “about” one particular value, and/or to “about” another particular value. When such a range is expressed, another embodiment comprises from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance can or can not occur, and that the description comprises instances where said event or circumstance occurs and instances where it does not.

“LifeStyle Management System (‘LMS’)” means a process or system that is utilized to associate a unique identifier associated with a consumable product to the nutritional content of the product for the purpose of monitoring, tracking and analyzing a dietary or nutritional regimen.

“LifeStyle Management Database” is a database of unique identifiers representing consumable products and the nutrition information associated with each unique identifier.

“Consumable products” comprises, for example and not meant to be limited to, prepackaged foods, prepared foods, home-cooked foods, recipes and produce.

A “barcode” is a precise arrangement of parallel lines (bars) and spaces that vary in width to represent data. A “UPC” or “Universal Product Code” is a standard for encoding a set of lines and spaces that can be scanned and interpreted into numbers to identify a product.

A “LifeStyle Management System LMS Barcode” is a barcode assigned by the LifeStyle Management System Database Owner to an item not historically having a UPC such a recipes, restaurant foods, home-cooked meals and produce or an aggregate grouping of these items.

“Consumption” is the intake of items into an animal's digestive system. Similarly, “nutritional intake” is the nutrient data corresponding to consumption.

The present invention can be understood more readily by reference to the following detailed description of preferred embodiments of the invention and to the Figures and their previous and following description.

In one embodiment of the present invention, a system and method for lifestyle management is provided. In one aspect, the system comprises a lifestyle selection unit for receiving a user selected lifestyle, a scanner for reading and storing a unique identifier associated with a product consumed, a unique identifier upload unit for transferring the unique identifier from the scanner to a user database, a nutrition database comprising nutritional information associated with a plurality of consumable products wherein each consumable product has an associated unique identifier, a nutrition information association unit for retrieving nutrition information of a consumed product from the nutrition database based on the unique identifier in the user database, and a nutritional monitor unit for providing dietary feedback to a user based on the user selected lifestyle in relation to the product consumed.

As will be described in more detail below, the lifestyle can be exemplarily selected from weight loss, weight gain, and weight maintenance. In varying aspects, the lifestyle selection unit can be configured to determine a goal weight, determining a time frame for reaching the goal weight, and determining an average caloric intake required to reach the goal weight.

The system can further comprise a meal creation unit wherein a user can select at least one serving (or a fraction thereof) of at least one scanned consumable product to comprise a meal. The system can further comprise a bar code generator for creating a bar code representing a meal or a consumable product. The nutritional monitor unit can be configured to perform analysis and to display the analytic results. Exemplary displays comprise, but are not limited to: displaying nutritional intake over time, displaying user measurements entered by the user, displaying nutritional intake over time against the user selected lifestyle, displaying measurements entered by the user the user selected lifestyle, and the like.

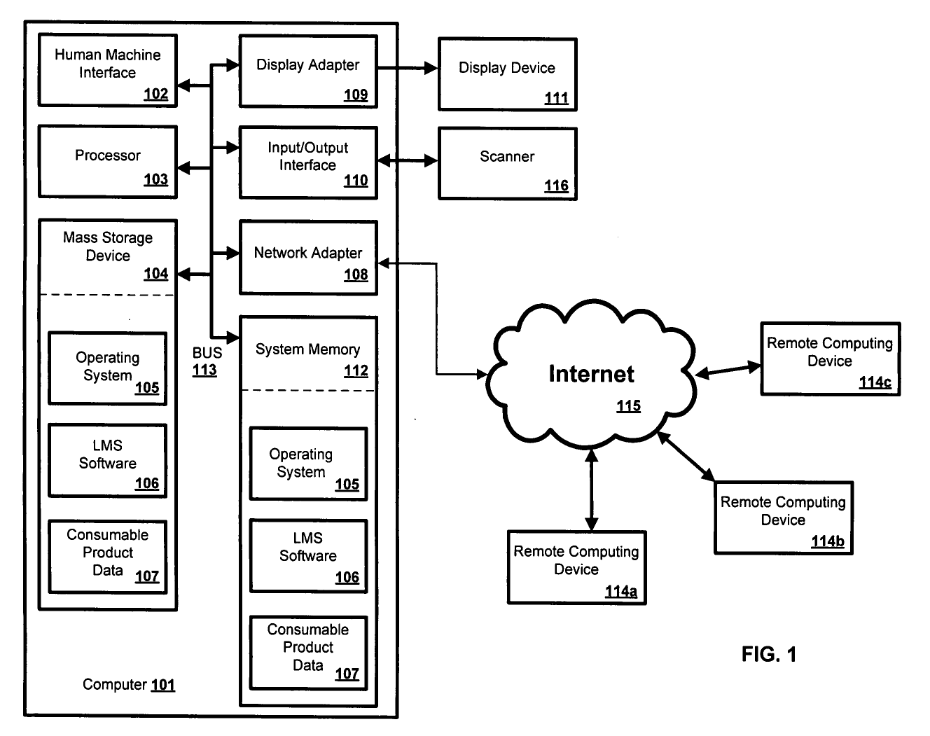

The system has been described above as comprised of units. One skilled in the art will appreciate that this is a functional description and that the respective functions can be performed by software, hardware, or a combination of software and hardware. A unit can be software, hardware, or a combination of software and hardware. The units can comprise the LMS Software 106 as illustrated in FIG. 1 and described below. In one exemplary aspect, the units can comprise a computer 101 as illustrated in FIG. 1 and described below.

FIG. 1 is a block diagram illustrating an exemplary operating environment for performing the disclosed method. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The system and method of the present invention can be operational with numerous other general purpose or special purpose computing system environments or configurations. Examples of well known computing systems, environments, and/or configurations that can be suitable for use with the system and method comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

In another aspect, the system and method of the present invention can be described in the general context of computer instructions, such as program modules, being executed by a computer. Generally, program modules comprise routines, programs, objects, components, data structures, etc. that perform particular tasks or implement particular abstract data types. The system and method of the present invention can also be practiced in distributed computing environments where tasks are performed by remote processing devices that are linked through a communications network. In a distributed computing environment, program modules can be located in both local and remote computer storage media including memory storage devices.

Further, one skilled in the art will appreciate that the system and method disclosed herein can be implemented via a general-purpose computing device in the form of a computer 101. The components of the computer 101 can comprise, but are not limited to, one or more processors or processing units 103, a system memory 112, and a system bus 113 that couples various system components including the processor 103 to the system memory 112.

The system bus 113 represents one or more of several possible types of bus structures, including a memory bus or memory controller, a peripheral bus, an accelerated graphics port, and a processor or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, and a Peripheral Component Interconnects (PCI) bus also known as a Mezzanine bus. The bus 113, and all buses specified in this description can also be implemented over a wired or wireless network connection and each of the subsystems, including the processor 103, a mass storage device 104, an operating system 105, LMS (Lifestyle Management System) software 106, consumable product data 107, a network adapter 108, system memory 112, an Input/Output Interface 110, a display adapter 109, a display device 111, and a human machine interface 102, can be contained within one or more remote computing devices 114a,b,c at physically separate locations, connected through buses of this form, in effect implementing a fully distributed system.

The computer 101 typically comprises a variety of computer readable media. Exemplary readable media can be any available media that is accessible by the computer 101 and comprises, for example and not meant to be limiting, both volatile and non-volatile media, removable and non-removable media. The system memory 112 comprises computer readable media in the form of volatile memory, such as random access memory (RAM), and/or non-volatile memory, such as read only memory (ROM). The system memory 112 typically contains data such as consumable product data 107 and/or program modules such as operating system 105 and LMS software 106 that are immediately accessible to and/or are presently operated on by the processing unit 103.

In another aspect, the computer 101 can also comprise other removable/non-removable, volatile/non-volatile computer storage media. By way of example, FIG. 1 illustrates a mass storage device 104 which can provide non-volatile storage of computer code, computer readable instructions, data structures, program modules, and other data for the computer 101. For example and not meant to be limiting, a mass storage device 104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 104, including by way of example, an operating system 105 and LMS software 106. Each of the operating system 105 and LMS software 106 (or some combination thereof) can comprise elements of the programming and the LMS software 106. Consumable product data 107 can also be stored on the mass storage device 104. Consumable product data 107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 101 via an input device (not shown). Examples of such input devices comprise, but are not limited to, a keyboard, pointing device (e.g., a “mouse”), a microphone, a joystick, a serial port, a scanner, and the like. These and other input devices can be connected to the processing unit 103 via a human machine interface 102 that is coupled to the system bus 113, but can be connected by other interface and bus structures, such as a parallel port, game port, or a universal serial bus (USB).

In yet another aspect of the present invention, a display device 111 can also be connected to the system bus 113 via an interface, such as a display adapter 109. It is contemplated that the computer 101 can have more than one display adapter 109 and the computer 101 can have more than one display device 111. For example, a display device can be a monitor, an LCD (Liquid Crystal Display), or a projector. In addition to the display device 111, other output peripheral devices can comprise components such as speakers (not shown) and a printer (not shown) which can be connected to the computer 101 via Input/Output Interface 110.

The scanner 116 can communicate with computer 101 via Input/Output Interface 110 or across a local or remote network. In one aspect, users utilize a scanner that is capable of collecting and storing unique identifier data. It will be appreciated that the scanner 116 can be any type of unique identifier scanning device, for example and not meant to be limiting, a bar code scanner, a camera, an RFID reader, and the like. In another aspect, the scanner 116 can be an independent stand alone device, or can be integrated into another device, such as a phone, music player, and the like. Optionally, the communication with computer 101 via Input/Output Interface 110 can be via a wired or wireless connection.

The computer 101 can operate in a networked environment using logical connections to one or more remote computing devices 114a,b,c. By way of example, a remote computing device can be a personal computer, portable computer, a server, a router, a network computer, a peer device or other common network node, and so on. Logical connections between the computer 101 and a remote computing device 114a,b,c can be made via a local area network (LAN) and a general wide area network (WAN). Such network connections can be through a network adapter 108. A network adapter 108 can be implemented in both wired and wireless environments. Such networking environments are conventional and commonplace in offices, enterprise-wide computer networks, intranets, and the Internet 115.

For purposes of illustration, application programs and other executable program components such as the operating system 105 are illustrated herein as discrete blocks, although it is recognized that such programs and components reside at various times in different storage components of the computing device 101, and are executed by the data processor(s) of the computer. An implementation of LMS software 106 can be stored on or transmitted across some form of computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any method or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The processing of the disclosed system and method of the present invention can be performed by software components. The disclosed system and method can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed method can also be practiced in grid-based and distributed computing environments where tasks are performed by remote processing devices that are linked through a communications network. In a distributed computing environment, program modules can be located in both local and remote computer storage media including memory storage devices.

In operation, the scanner can be used to collect and store the unique identifiers on consumable products, such as, for example, bar codes found on food items. In another aspect, the stored unique identifiers can be uploaded to a secure online user account. In one aspect, the barcode can be associated with the food items' specific nutritional information, which can then be used to update the user's profile. In a further aspect, the user can utilize analysis tools to monitor the foods to be consumed and the associated nutritional content. In another aspect, the users can compare the nutritional intake of the foods to be consumed to customizable goals or benchmarks. In other various aspects, the user can input, monitor and track other health-related items such as amount of exercise, weight, body measurements, blood pressure and blood sugar levels.

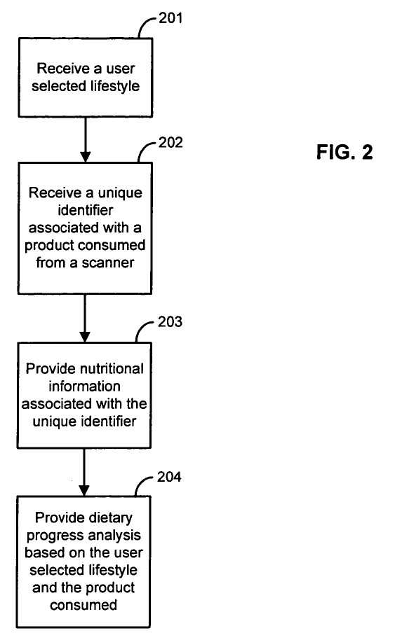

As shown in FIG. 2, disclosed is a method for lifestyle management comprising receiving a user selected lifestyle at block 201, receiving a unique identifier associated with a product consumed from a scanner at block 202, providing nutritional information associated with the unique identifier at block 203, and providing dietary progress analysis based on the user selected lifestyle and the product consumed at block 204. In various aspects, the lifestyle selected by the user can be weight loss, weight gain, or weight maintenance.

The method can further comprise the user selecting at least one serving (or a fraction thereof) of at least one scanned consumable product to comprise a meal. The method can further comprise receiving the user selected at least one serving of at least one scanned consumable product. In another aspect, the method can comprise generating a bar code representing a meal or a consumable product. The dietary progress analysis provided can be, for example and not meant to be limiting, a display of nutritional intake over time, a display of user measurements entered by the user, a display of nutritional intake over time against the user selected lifestyle, and/or a display of measurements entered by the user the user selected lifestyle. One skilled in the art will appreciate that the display can be in any conventional format, such as, for example and not meant to be limiting, a chart, an interactive display, and the like.

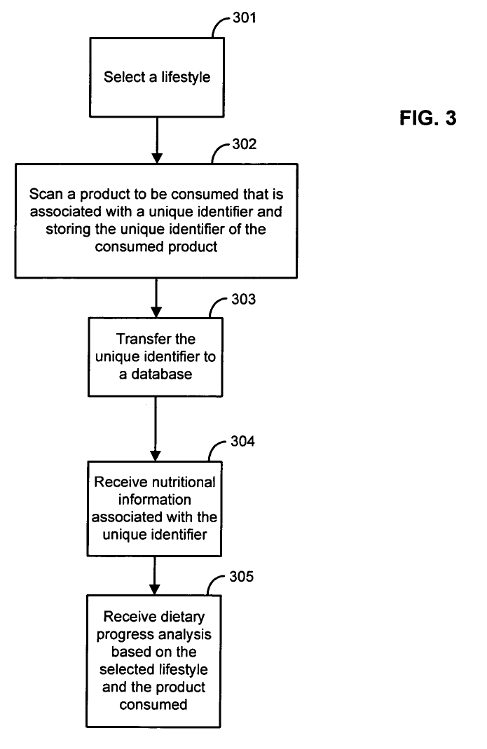

Referring now to FIG. 3, a method for lifestyle management is illustrated that comprises selecting a lifestyle at block 301; scanning a product to be consumed and storing a unique identifier associated with the product to be consumed at block 302; and transferring the unique identifier to a database at block 303. In other aspects, the method can further comprise receiving nutritional information associated with the unique identifier at block 304 and receiving dietary progress analysis based on the selected lifestyle and the product consumed at block 305. As noted above, in exemplary aspects, the lifestyle can be weight loss, weight gain, or weight maintenance.

The lifestyle management method can further comprise selecting at least one serving (or a fraction thereof) of at least one scanned consumable product to comprise a meal. In operation, a bar code is generated that represents a meal or a consumable product. In this aspect, the bar code can be printed. As noted above, the provided dietary progress analysis can be, for example and not meant to be limiting, a display of nutritional intake over time, a display of user measurements entered by the user, a display of nutritional intake over time against the user selected lifestyle, or a display of measurements entered by the user the user selected lifestyle.

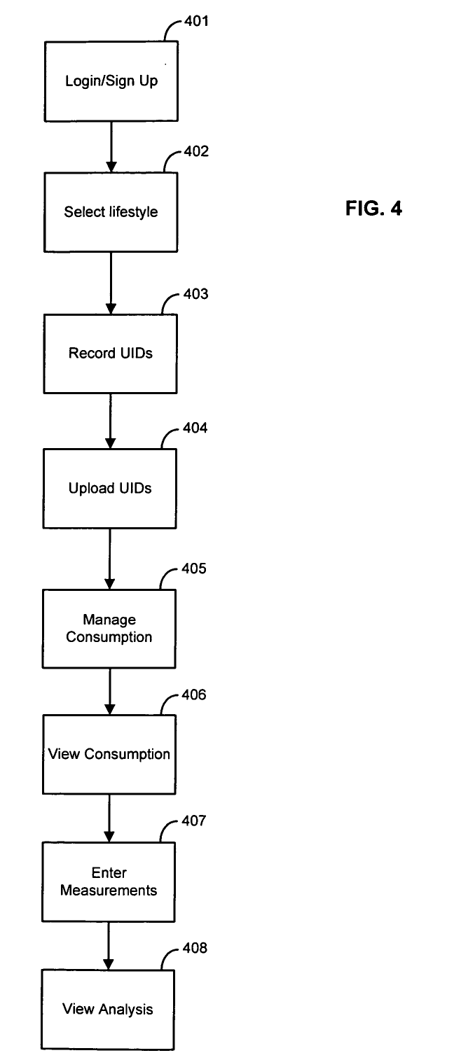

FIG. 4 is a flowchart illustrating an exemplary method of the present invention. At block 401 a user can login to the system, or if not already possessed of an account, the user can sign up for a new account. The signup process can comprise, for example and not meant to be limiting, creating a username and password, entering billing information, entering health profile information, and the like. The health profile information can exemplarily comprise, but is not limited to, the following: Birth date (used to derive current age); Weight; Gender; Physical Activity Level (such as, for example, sedentary (i.e., little or no exercise), lightly active (i.e., light exercise/sports 1-3 days/week), moderatetely active (i.e., moderate exercise/sports 3-5 days/week), very active (i.e., hard exercise/sports 6-7 days a week, and extra active (i.e., very hard exercise/sports & physical job or 2× training)); Body Frame Size; Primary Weight Goal (such as, for example, lose body weight, maintain current body weight, or gain body weight); Current Medical Conditions; and/or Any Diet Regimen the user is currently participating in.

At block 402, the user can select a lifestyle by setting various goals. These goals can exemplarily comprise, but are not limited to, determining a goal weight (this can comprise maintaining current weight), determining a time frame for reaching the goal weight, and/or determining an average caloric intake required to reach the weight goal.

For example, a user can select a weight goal (if any) and a time frame in which to reach that weight goal (if any). In one aspect, if the user does not have a weight loss or gain goal (i.e., the lifestyle selected is weight maintenance), the system can display the calories/time, for example, a daily caloric intake, required to maintain current weight.

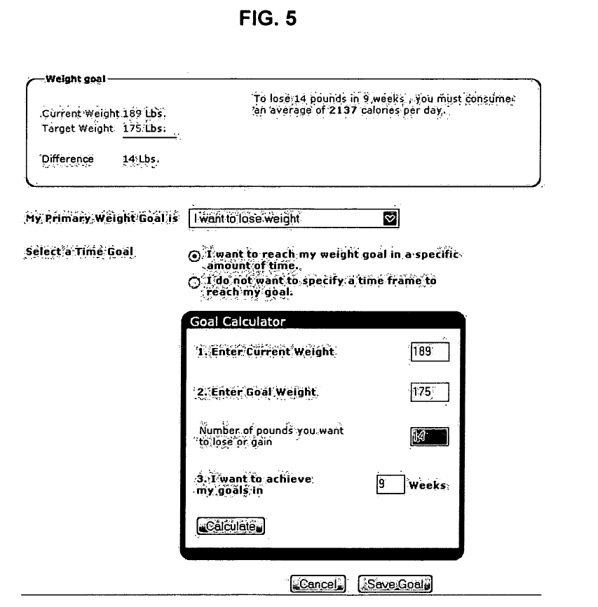

In a further aspect, if the user elects to lose weight, the user can select a time goal or decline a time goal, which determines which Weight Goal Calculator is presented to the user. For example, if the user elected to reach a weight goal in a specific amount of time, a first Weight Goal Calculator can be presented in which the user's current weight can be pre-populated with the current weight entered during signup, or the last weight entered by the user. In this aspect, all the fields can be populated with the values calculated the last time the calculator was used if the user has used the calculator before. In one aspect, the user can enter a goal weight or the number of pounds they want to lose or gain and the number of weeks in which to achieve the goal. Based off this information, the first Weight Goal Calculator determines the user's caloric needs and displays the results. It is contemplated that the user is only required to enter one of either the Goal Weight or pounds to be lost as the first Weight Goal Calculator is programmed to calculate and display the non-entered value. In this aspect, the first Weight Goal Calculator determines the caloric needs to achieve the goal weight. An exemplary user interface for the first Weight Goal Calculator is shown in FIG. 5.

If the user does not specify a time frame for the selected lifestyle, then a second Weight Goal Calculator can be presented. The current weight entry of the second Weight Goal Calculator can be pre-populated with the current weight of the user that was entered during signup, or the last weight entered by the user. Of course, if the user has used the calculator before, it is contemplated that all the fields can be populated with the values calculated the last time the second Weight Goal Calculator was used. In this aspect, the user can enter either the Goal Weight or the number of pounds they want to lose or gain and the second Weight Goal Calculator will calculate and display the non-selected value. The second Weight Goal Calculator subsequently determines the caloric needs to maintain the goal weight.

At block 403 of FIG. 4, the user can record unique identifiers (UIDs), such as barcodes. In this aspect, the user can utilize a portable scanner to collect and record unique identifier data. Additionally, the time at which the barcode data was collected can be recorded. At block 404, the user can upload the scanned unique identifiers into the system. In various aspects, the uploaded unique identifiers can be stored locally on a user computer, or on a remote computer. In another aspect, the user can upload and edit barcode information and scanned times. In a further aspect, the user can select and edit items that were consumed that do not have an associated barcode such as, for example and not meant to be limiting, barcoded items that were eaten but the barcode data was not collected using the device, non-barcoded items that were consumed, restaurant items, fruits and vegetables, recipes, and user-defined meals.

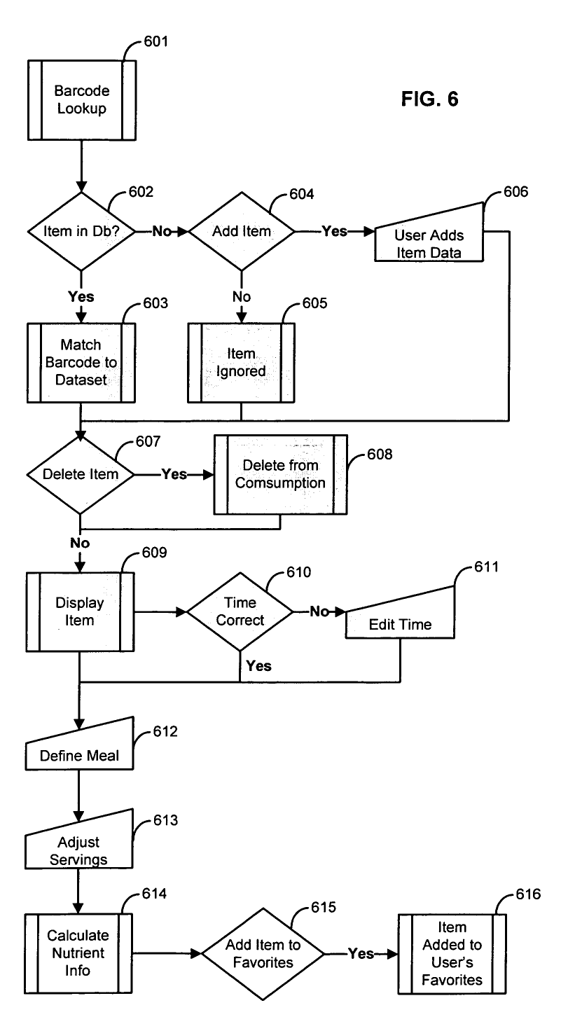

At block 405, the user can manage consumption. A more detailed flowchart representing exemplary consumption management is shown in FIG. 6 and is described below. In one aspect, the user can save items to the user's favorites list and delete items from consumption history. In other exemplary aspects, the user can view the items consumed for a given day, add items to consumption history for a given day, add items to the LifeStyle Management database, edit the number of servings consumed, edit the times of day that items were consumed, assign a consumed item to a specific meal (breakfast, lunch, brunch, snack, dinner), and add a consumed item to the user's favorite foods list.

In a further aspect of the present invention, the user can add a consumable product to the LMS database. In one aspect, the user can also add a consumable product to a favorites list. In this aspect, a user can flag a given consumable product, recipe or meal as one of the users favorites, which enables the user to reduce the amount of time and/or effort required to locate the consumable product in the future.

Additionally, the user can print barcodes. For example, the user can locate a consumable product in the LifeStyle Management database and print a corresponding barcode for that item. In this aspect, the user can also print barcodes for prepackaged food items, restaurant items, recipes, produce, and user defined meals.

As noted above, the user can create meals in the system of the present invention. In one aspect, the user can create a meal by selecting items from the LifeStyle Management database and adding the items to a user defined meal. This allows the user to optionally view the aggregate nutritional content of the meal and save the meal under a user-defined name. Optionally, the user can generate an LMS Barcode for the meal and associate the LMS Barcode to the saved meal.

In another aspect, a user can manage consumption as shown in FIG. 6. The manage consumption process enables a user to match the unique identifiers associated with consumable products, such as barcodes, to the nutritional information associated with the consumable product in the LMS Database, edit the items and add items nonexistent in the global database. The exemplary method will be described in the context of barcodes, however, one skilled in the art will appreciate that this context is merely exemplary and is non-limiting.

In this aspect, at block 601, the user can perform a barcode lookup. The barcode data previously uploaded is compared to the LMS Database to determine if the barcode and corresponding nutritional information exist for the particular barcode. If it is determined that the barcode and corresponding nutritional information are in the LMS Database at block 602, the barcode data is used to determine the applicable nutritional information at block 603 and the consumable product can be added to the user's consumption history.

However, if it is determined, at block 602, that the barcode and corresponding nutritional information is not in the LMS Database, the user can be provided with the option to add the data at block 604. If the data is not entered at block 604, the barcode is ignored (not considered in the user's consumption history) and the system goes to block 607. If the data is entered at block 604, the barcode and associated nutritional information is stored and the system goes to block 607.

At block 607, the user can be provided with the option to delete an item from consumption history. If the user elects to do so, the system can remove the item at block 608. At block 609, the barcode associated with a consumable product, the number of servings consumed (defaulted to 1), the date/time of the barcode scan and the meal (i.e., breakfast, lunch, brunch, snack, dinner) during which the item was consumed can be displayed. This can be displayed, for example, via a web-based graphical user interface (GUI).

At block 609, the date/time of the barcode scan is interpreted as the time at which the user consumed the item. The user has the option at block 610 to update the date/time to correspond to the actual time of consumption. If the user chooses to do so, the system can adjust the date/time at block 611.

At block 612, the user can define a meal and can specify during which meal the item was consumed (i.e., breakfast, lunch, brunch, snack, dinner). Then, at block 613, the user can adjust the servings consumed during the meal. In one aspect, the number of servings consumed can be assumed by the system to be one per consumable product. Optionally, the user can update the number of servings to correspond to the actual number of servings consumed.

In another aspect, the nutrient information can be calculated at block 614. In this aspect, the nutrient information for the item consumed can be multiplied by the number of servings consumed to derive the nutritional intake consumed. For example:
Given: Calories per serving=100; Servings=2
Result: Calories Consumed=2*100=200 calories

At block 615, the user can optionally select one or more items to be added to the user's favorites. This flags the consumed products to enable them to be searched for and found more easily. The system can add the consumed product at block 616.

Returning to the flowchart illustrated in FIG. 4, at block 406 the user can optionally view consumption history, which allows a user to view the items consumed for a given range of days and corresponding nutritional information. In other exemplary aspects, the user can view aggregate nutritional totals for items consumed for a range of days, edit the number of servings consumed of items, add consumed items to the user's favorite foods list, and delete an item from the consumption history.

At block 407, the user can optionally enter the user's measurements into the system. This enables a user to enter and save health related and physical measurements for a given day. Examples of the user's measurements comprise, but are not limited to: body weight, blood pressure, hip size, waist size, forearm size, body frame, height, age, physical activity level, and the like.

At block 408, a user can view a display of an analysis of the user's progression. For example, the user can view graphs that represent how well the user is adhering to the selected lifestyle. In other various aspects, the user can, for example and not meant to be limiting, view and save charts displaying nutritional intake over a time specified by the user, view and save charts displaying measurements entered by the user, view and save charts displaying nutritional intake over time against user defined goals, and view and save charts displaying measurements entered by the user against user defined goals.

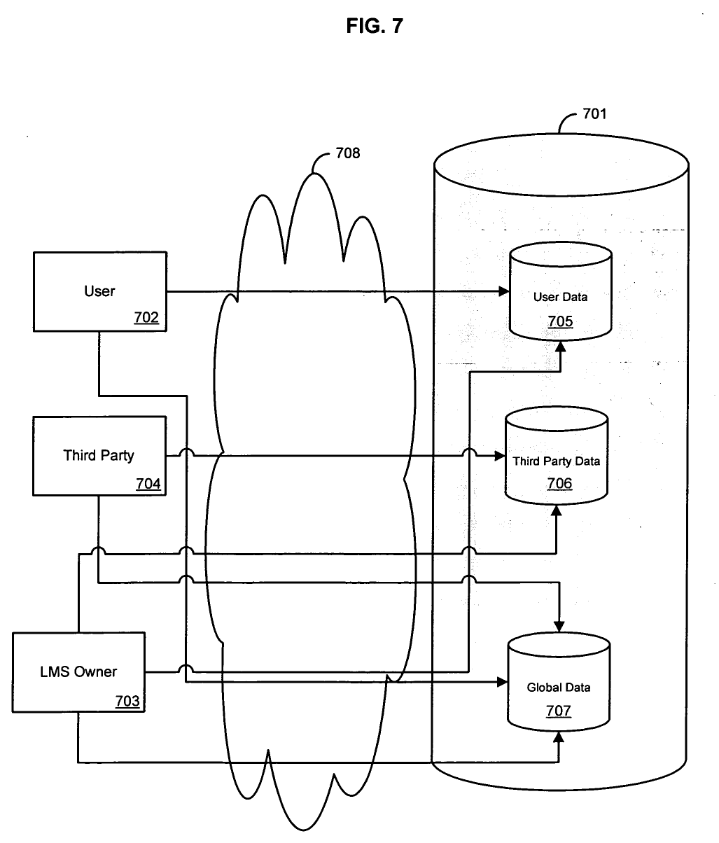

FIG. 7 illustrates exemplary data relationships of the present invention. Unique identifier and associated nutritional data stored in the LifeStyle Management Database 701 can be collected and entered by numerous entities. Optionally, this data entry can be directly into the LifeStyle Management Database 701, or via a network 708. Exemplary data collectors can comprise users 702, a LifeStyle Management Database owner 703, and third parties 704.

In one aspect, a user 702 can add a consumable product and its associated unique identifier and nutritional information to the LMS Database 701 by way of a user database 705. Once the data is added by the user 702, the data can be utilized by that specific user 702. It is contemplated that other users 702 can either be given access to the data, or be restricted from accessing the data.

In a further aspect of the system, a third party 704, such as an entity who is not a user 702 of the LifeStyle Management System, can add a consumable product and its associated unique identifier and nutritional information to the LMS Database 701 by way of a third party database 706. Further, the LMS Database Owner 703 can add a consumable product and its associated unique identifier and nutritional information to the LMS Database 701. It is contemplated that the LMS Database Owner 703 can review the data added by users 702 and third parties 704. In this aspect, the data can be checked for accuracy and edited if needed. If the data added by users and third parties is does not have a barcode, an LMS Barcode can be created and added to the item's dataset. In another aspect, the LMS Database Owner 703 can add data to the user database 705, the third party database 706, and the global database 707.

Optionally, once a consumable product has been added to the global database 707 it can be made accessible by all LMS users 702 and third parties 704. This use can comprise, but is not limited to, searching the LMS Database for items and printing the associated barcodes. The barcodes can be, for example, a UPC or LMS barcode.

While this invention has been described in connection with preferred embodiments and specific examples, it is not intended that the scope of the invention be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A system for lifestyle management, comprising:
a lifestyle selection unit for receiving a user selected lifestyle;
a scanner for reading and storing a unique identifier associated with a product consumed;
a unique identifier upload unit for transferring the unique identifier from the scanner to a user database;
a nutrition database comprising nutritional information associated with a plurality of consumable products wherein each consumable product has an associated unique identifier;
a nutrition information association unit for retrieving nutrition information of a consumed product from the nutrition database based on the unique identifier in the user database that is associated with the product consumed; and
a nutritional monitor unit for providing dietary feedback to a user based on the user selected lifestyle in relation to the product consumed.

2. The system of claim 1, wherein the user selected lifestyle selected is selected from the group consisting of weight maintenance; weight loss; and weight gain.

3. The system of claim 1, wherein the lifestyle selection unit is configured for determining a goal weight; determining a time frame for reaching the goal weight; and determining an average caloric intake required to reach the goal weight.

4. The system of claim 1, wherein the scanner is selected from the group consisting of a bar code scanner; a camera; and an RFID reader.

5. The system of claim 1, further comprising a meal creation unit, wherein a user can select at least one serving of at least one scanned consumable product to comprise a meal.

6. The system of claim 1, further comprising a bar code generator for creating a bar code representing a meal.

7. The system of claim 1, wherein the nutritional monitor unit is configured for generating a chart displaying nutritional intake over time.

8. The system of claim 1, wherein the nutritional monitor unit is configured for generating a chart displaying user measurements entered by the user.

9. The system of claim 1, wherein the nutritional monitor unit is configured for generating a chart displaying nutritional intake over time against the user selected lifestyle.

10. The system of claim 1, wherein the nutritional monitor unit is configured for generating a chart displaying measurements entered by the user.

11. A method for lifestyle management, comprising:
receiving a user selected lifestyle;
receiving a unique identifier from a scanner wherein the unique identifier is associated with a product consumed;
providing nutritional information associated with the unique identifier; and
providing dietary progress analysis based on the user selected lifestyle and the product consumed.

12. The method of claim 11, wherein the user lifestyle selected is selected from the group consisting of weight maintenance; weight loss; and weight gain.

13. The method of claim 11, wherein the scanner is selected from the group consisting of a bar code scanner; a camera; and an RFID reader.

14. The method of claim 11, further comprising receiving a user selected at least one serving of at least one scanned consumable product to comprise a meal.

15. The method of claim 11, further comprising generating a bar code representing a meal.

16. The method of claim 11, wherein the dietary progress analysis provided is selected from the group consisting of a display of nutritional intake over time; a display of user measurements entered by the user; a display of nutritional intake over time against the selected lifestyle; and a display of measurements entered by the user.

17. A method for lifestyle management, comprising:
selecting a lifestyle;
scanning a product consumed wherein a unique identifier associated with the product is stored;
transferring the unique identifier to a database;
receiving nutritional information associated with the unique identifier; and
receiving dietary progress analysis based on the selected lifestyle and the product consumed.

18. The method of claim 17, wherein the lifestyle selected is selected from the group consisting of weight maintenance; weight loss; and weight gain.

19. The method of claim 17, wherein the scanner is selected from the group consisting of a bar code scanner; a camera; and an RFID reader.

20. The method of claim 17, further comprising selecting at least one serving of at least one scanned consumable product to comprise a meal.

21. The method of claim 17, further comprising generating a bar code representing a meal.

22. The method of claim 17, further comprising printing a bar code.

23. The method of claim 17, wherein the dietary progress analysis received is selected from the group consisting of a display of nutritional intake over time; a display of user measurements entered by the user; a display of nutritional intake over time against the user selected lifestyle; and a display of measurements entered by the user.
